(19) United States
(12) Patent Application Publication
(10) Pub. No.: US 2012/0215133 A1
(43) Pub. Date: Aug. 23, 2012

(54) CATHETER TIP DEVICE AND METHOD FOR MANUFACTURING SAME

(76) Inventors: Brad Jeffrey Neiman, Fremont, CA (US); Mark Anthony Sheward, Fremont, CA (US); Calin Victor Miclaus, Fremont, CA (US); Joseph Francis Mulqueen, Fremont, CA (US)

(21) Appl. No.: 13/032,038

(22) Filed: Feb. 22, 2011

(51) Int. Cl. A61B 5/05; H05K 7/02; H05K 13/00

(52) U.S. Cl. 600/585; 29/592.1; 361/807

(57) ABSTRACT

A catheter tip device and methods for manufacturing of a catheter tip device. The device comprises a transducer module attached to a capsule, wherein the transducer module comprises a carrier. The carrier includes a recessed die-attach area, a lead area having at least one groove disposed therein and includes a barrier. The barrier extends from the carrier to partially circumscribe the recessed-die attach area. The methods of manufacturing the catheter tip device involve the use of inserting the carrier into the capsule.

### BACKGROUND OF THE INVENTION

The subject matter disclosed herein generally relates to catheters, and more particularly to a catheter tip device.

Catheter tip devices are widely used in the medical diagnostics field for carrying various components, including an integrated circuit die, mounted in a capsule of a catheter tip. For example, a sensor die mounted within a catheter tip is insertable into a living body through a body orifice and/or through a surgical incision. The components and construction of at least some known catheter tip devices generally require that much of the manufacturing and/or assembly process be performed manually. For example, known circuit dies are manually coupled to electrical connections and then are manually mounted within the capsule via a carrier. Accurately mounting the carrier/die within the capsule enhances operation of the catheter tip device and facilitates placement of the catheter tip device within the body. However, placement of the die within the capsule is often difficult or inaccurate and may be dependant on the technician assembling the catheter tip device. Often, the die placement within the capsule results in the electrical connection of the die contacting the capsule resulting in operational problems and/or failure of the die. Moreover, the use of manual assembly generally increases manufacturing costs and/or human errors associated with such manufacture.

### BRIEF DESCRIPTION OF THE INVENTION

In one aspect, a transducer module is provided. The transducer module includes a carrier and a barrier extending from the carrier to facilitate protecting a die area of the carrier during manufacturing processes.

In another aspect, a catheter tip device is provided. The catheter tip device includes a capsule, a transducer module and a barrier extending from the carrier to facilitate protecting a die area of the carrier during manufacturing processes.

In a further aspect, a method of manufacturing a catheter tip device is provided. The method includes forming a carrier including a barrier that facilitates protecting an area of the carrier, and includes forming a grooved lead area on the carrier that facilitates receiving a conductive lead.

### BRIEF DESCRIPTION OF THE DRAWINGS

FIG. 1 illustrates a perspective view of an exemplary catheter tip device.

FIG. 2 illustrates a plan view of the catheter tip device shown in FIG. 1.

FIG. 3 illustrates a plan view of a transducer module that may be used with the catheter tip device shown in FIG. 1.

FIG. 4 illustrates a partial, cross-sectional view of the catheter tip device shown in FIG. 1 and taken along lines 4-4 of FIG. 3.

FIG. 5 illustrates a side view of the transducer module shown in FIG. 3.

FIG. 6 illustrates a partial end view of the transducer module shown in FIG. 3.

FIG. 7 illustrates a flowchart of an exemplary method for use in manufacturing the catheter tip device shown in FIG. 1.

### DETAILED DESCRIPTION OF THE INVENTION

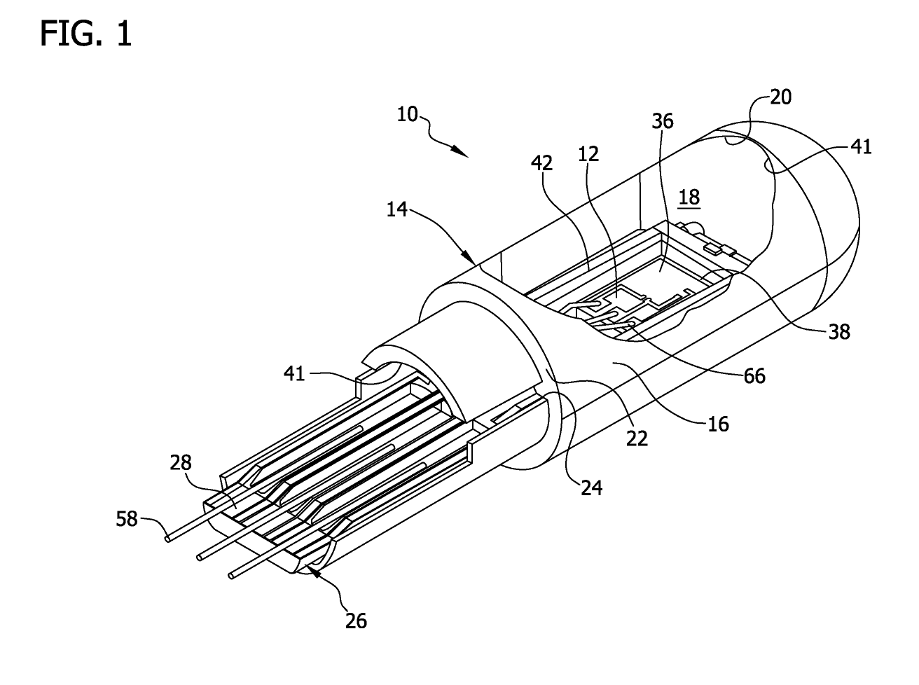

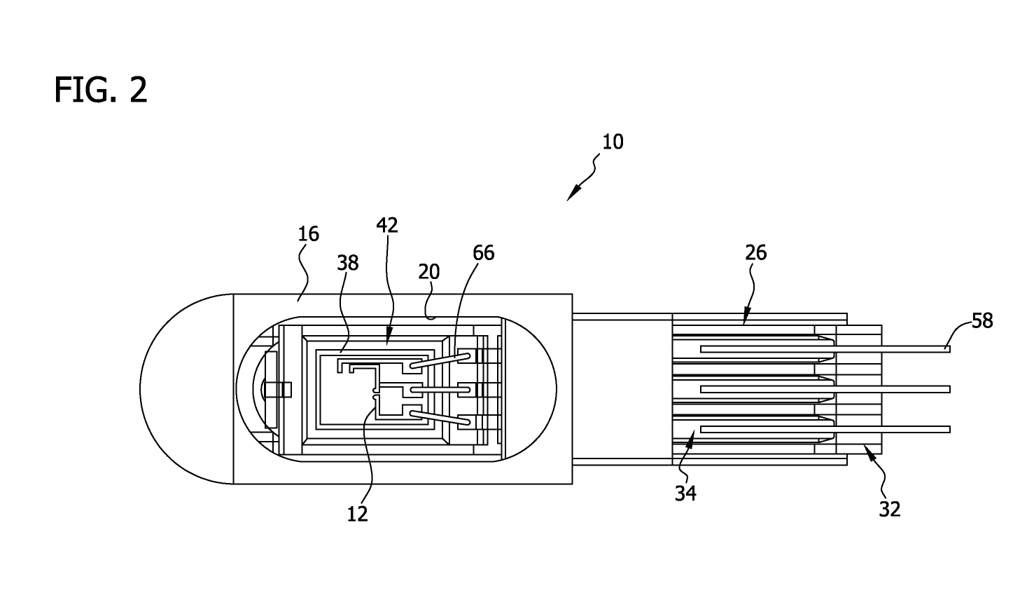

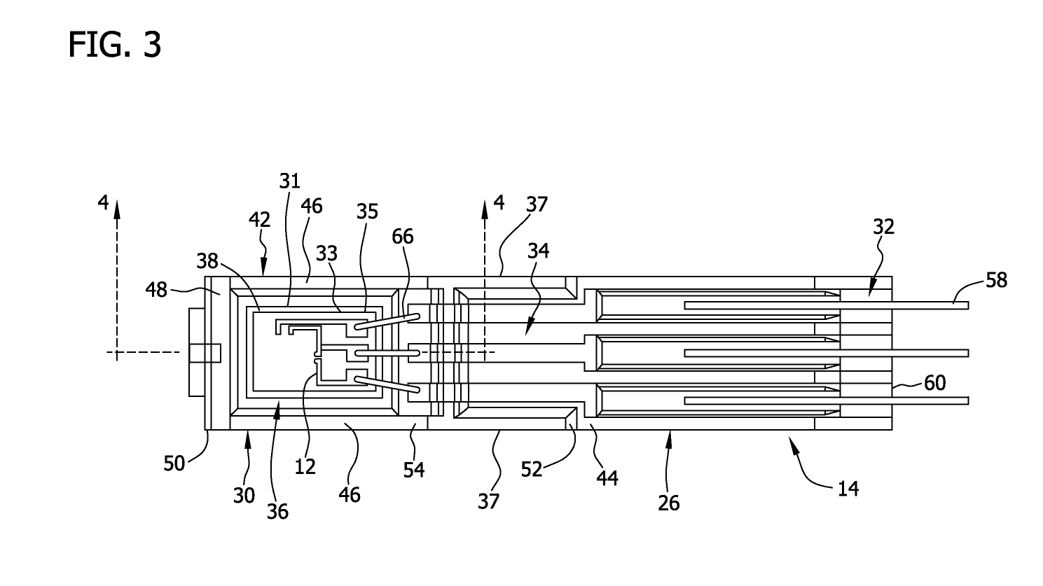

FIG. 1 is a perspective view of an exemplary catheter tip device 10. FIG. 2 is a plan view of device 10. FIG. 3 is a plan view of a transducer module 14 used with device 10. In the exemplary embodiment, catheter tip device 10 is used to carry one or more integrated circuit dies 12 mounted in a catheter tip (not shown). For example, die 12 may be, but is not limited to being, for use with transducers (for example, sensors and actuators), data processing devices (for example, ASIC microprocessors), and/or telemetric devices (for example, for wireless or RF communication). The dies 12 can be configured to provide an electrical signal output in response to an external boundary condition (for example, pressure, temperature, pH, etc.) of a patient. Device 10 can be insertable into a living body (not shown) through an orifice in the body and/or through a surgical incision, and can be used for a variety of applications, including, for example, to perform direct measurements, within the body, of parameters such as pressure, temperature, pH, etc. In the exemplary embodiment, catheter tip device 10 includes a transducer module 14 that is coupled to a capsule 16.

Capsule 16 can be fabricated from, but is not limited to being fabricated from, a bio-compatible material such as, but not limited to, a plastic material. For example, capsule 16 is fabricated from an ISO 10993-compliant material. One skilled in the art will appreciate that other medical-grade materials could be used to fabricate capsule 16, including, for example, metal, ceramic, or composite materials. In the exemplary embodiment, capsule 16 is substantially cylindrical and includes a cavity 18 defined therein that is sized and oriented to receive at least a portion of transducer module 14 therein. Alternatively, capsule 16 may have any other shape, size and/or orientation that enables catheter tip device 10 to function as described herein. Capsule 16 also includes a window 20 that enables at least a portion of transducer module 14 to be viewed. In the exemplary embodiment, capsule 16 includes a casing 22 having opposing grooves 24 that receive the transducer module 14.

In the exemplary embodiment, transducer module 14 includes at least one transducer die 12 coupled to a carrier 26. Transducer die 12 can be, for example, but is not limited to being, a micro-machined sensing or actuator element. Carrier 26 can be manufactured using molded interconnect device (MID) technology. In one embodiment, the carrier 26 is fabricated from a plastic material. In another embodiment, carrier 26 is fabricated from a ceramic material. It should be noted that one skilled in the art would appreciate carrier 26 may be fabricated from any material that enables carrier 26 to function as described herein. In the exemplary embodiment, carrier 26 includes a surface 28 formed with a die area 30, a lead area 32, and a lead pad area 34 that is between die and lead areas 30 and 32, respectively.

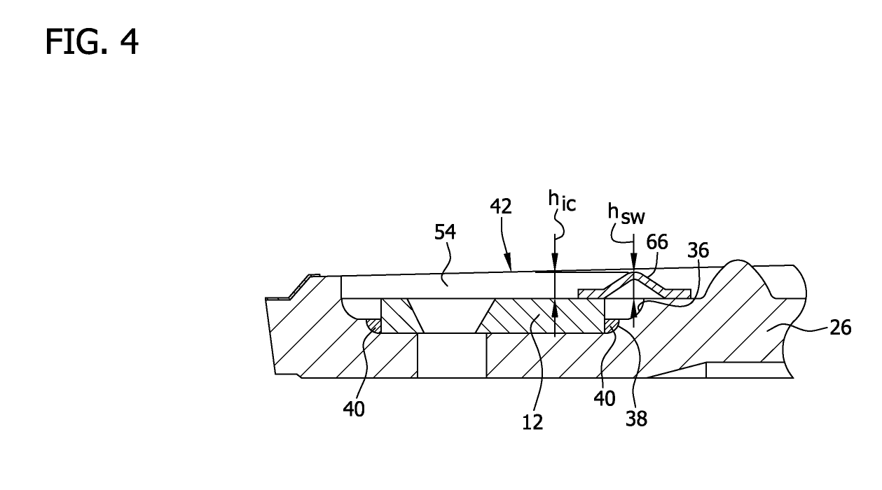

Carrier 26, in the exemplary embodiment, includes a recessed die-attach area or well 36 that has an outer perimeter 31 that is larger than an outer perimeter 33 of the transducer die 12 received within area 36. As such, an open groove 38 is formed between one or more edges 35 of transducer die 12 and outer perimeter of area 36. FIG. 4 illustrates a partial cross-sectional view of recessed die area 36 and groove 38. Recessed die-attach area 36 enables transducer die 12 to be inserted onto carrier 26. In the exemplary embodiment, die-attach area 36 is sized and oriented to receive transducer die 12 and at least one other device, such as, for example, a ASIC and/or a RF transceiver. In another embodiment, carrier 26 can include two or more recessed die-attach areas 36 that are sized and oriented to receive two or more dies 12. In another embodiment, the recessed-die attached area 36 does not include the groove 38 formed between edge 35 of die 12 and perimeter of area 36.

In the exemplary embodiment, transducer die 12 is coupled within recessed die-attach area 36 using an adhesive agent 40, such as a silicone gel or a Room Temperature Vulcanized (RTV) silicone applied inwardly from groove 38. Groove 38 facilitates maintaining agent 40 within area 36 and thus prevents the seepage of adhesive agent 40 out of recessed die-attach area 36.

To facilitate shielding recessed die-attach area 36 and to facilitate aligning transducer module 14, carrier 26 also includes a barrier 42 extending upward from an upper surface 44 of carrier 26. Barrier 42 includes opposing sidewalls 46 and an end wall 48. Each sidewall includes a first end 50, a second end 52 and a body 54 extending between first and second ends 50 and 52, respectively. Sidewalls 46 can be positioned at or near outer edges 37 of carrier upper surface 44. In the exemplary embodiment, each first end 50 is near carrier die area 30 and each second end 52 is within lead pad area 34. Alternatively, second end 52 can be positioned at carrier end 60. Regardless of the location of second end 52, body 54 extends between first end 50 and second end 52, and end wall 48 extends from each first end 50 of sidewalls 46. In such a configuration, end wall 48 and sidewalls 46 partially circumscribe recessed die-attach area 36 to facilitate protecting recessed die attached area 36 from handling during manufacturing processes. Moreover, in an embodiment, sidewalls 46 partially border lead pad area 34.

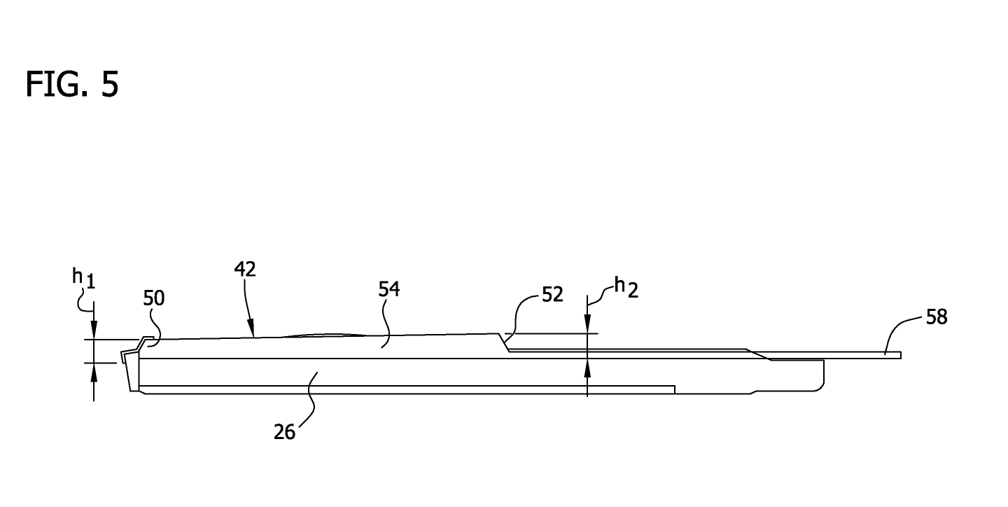

FIG. 5 is a side view of transducer module 14. In the exemplary embodiment, a height h₁ of sidewall first end 50 is shorter than a height h₂ of sidewall second end 52, such that body 54 tapers upwardly from first end 50 towards second end 52. In one embodiment, body 54 tapers at an angle less than, or approximately equal to 45° with respect to carrier upper surface 44. In another embodiment (not shown), first end 50 and second end 52 are formed with approximately the same height. Alternatively, first end 50 and/or second end 52 can be formed to cause body 54 to taper at an oblique angle towards carrier surface 44. In another embodiment, first end 50 and/or second end 52 are substantially straight parallel to carrier surface 44.

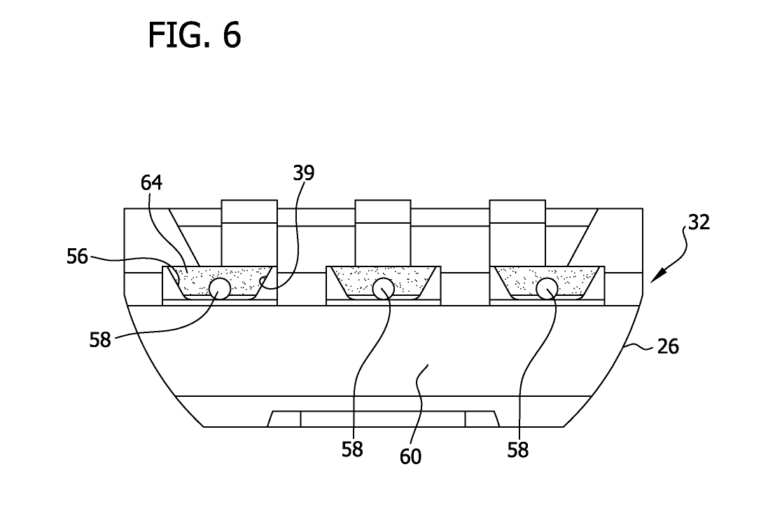

FIG. 6 is an end view of transducer module 14. In the exemplary embodiment, lead area 32 of carrier 26 includes at least one groove 56. Each groove 56 is sized and oriented to receive at least one conductive lead 58 therein. More specifically, in the exemplary embodiment, each groove 56 extends substantially axially from carrier end 60 towards lead pad area 34. In the exemplary embodiment, groove 56 includes angled and opposing walls 39 that are formed in a trough configuration that is oriented and sized to accept conductive lead 58 therein. Grooves 56 can be formed in any other shape, such as, but not limited to, a square form that enables conductive lead 58 to function as described herein.

Lead area grooves 56 facilitate aligning the conductive lead 58 to and/or within lead area 32. Grooves 56 also facilitate maintaining deposition material 64, such as, for example, solder material or conductive epoxy material therein, for use in coupling conductive lead 58 within groove 56. Moreover, grooves 56 facilitate reducing a likelihood of lead attach material 64 electrically bridging to an adjacent conductive lead 58.

In an aspect, one or more conductive leads 58 can be deposited on carrier 26, for example, via metal plating, as a substitute for a conventional printed circuit board. Conductive leads 58 can be used for interconnecting transducer die 12 to a device equipped to receive electrical signals transmitted from transducer die 12. Such conductive leads 58 are typically metallic. Alternatively, other materials can be used for fabricating conductive leads 58.

Transducer die 12 can be interconnected to one or more conductive leads 58. In one embodiment, an interconnect 66 (FIG. 4) between transducer die 12 and conductive leads 58 can be an electrical interconnect provided by one or more bond wires (not shown). The bond wires can be formed from fine wires having a diameter of, for example, 25 μm to 75 μm. The bond wires can be fabricated from materials such as, but not limited to, gold, aluminum, silver, or copper. One skilled in the art would appreciate that other wire materials can be used. In another embodiment, the electrical interconnect of transducer die 12 to conductive leads 58 can be provided by using flip-chip technology that includes solder bumps rather than bond wires. Interconnect 66 can be formed in any configuration that facilitates electrical connection as described herein.

In the exemplary embodiment, barrier 42 extends upward from carrier 26 beyond interconnects 66. As illustrated, sidewalls 46 extend above, and along, recessed die-attach area 36 and are adjacent to outermost interconnects 66. The heights h_{sw} of sidewalls 46 near interconnects 66 are taller than the heights h_{ic} of interconnects 66. As such, sidewalls 46 facilitate protecting interconnects 66 during assembly of transducer module 14 to capsule 16. Moreover, sidewalls 46 act as alignment guides or rails on outer edges 37 of carrier 26 to enable carrier 18 to be inserted within capsule grooves 24. Furthermore, sidewalls 46 facilitate maintaining carrier 26 approximately level with capsule 16 during carrier-to-capsule insert. In particular, sidewalls 46 facilitate preventing carrier 26 from pitching upward during insertion, which in turn, prevents interconnects 66 from contacting an inner portion 41 of capsule 16. Additionally, in the exemplary embodiment, sidewalls 46 and end wall 48 of barrier 42 facilitate preventing any protective coating or encapsulant or isolation material for sensor die 12 and interconnects 66 from being contacted by assemblers during assembly of device 10. Further, barrier 42 facilitates containment of any protective coating or encapsulant or isolation material during shipment and further handling of the device 10.

During manufacturing of catheter tip device 10, transducer module 14 can be attached to the capsule 16 in a variety of ways including, but not limited to, plastic welding, solvent bonding, and/or using an adhesive agent. Additionally, the capsule 16 can be filled with an encapsulant such as, for example, a dielectric silicone potting.

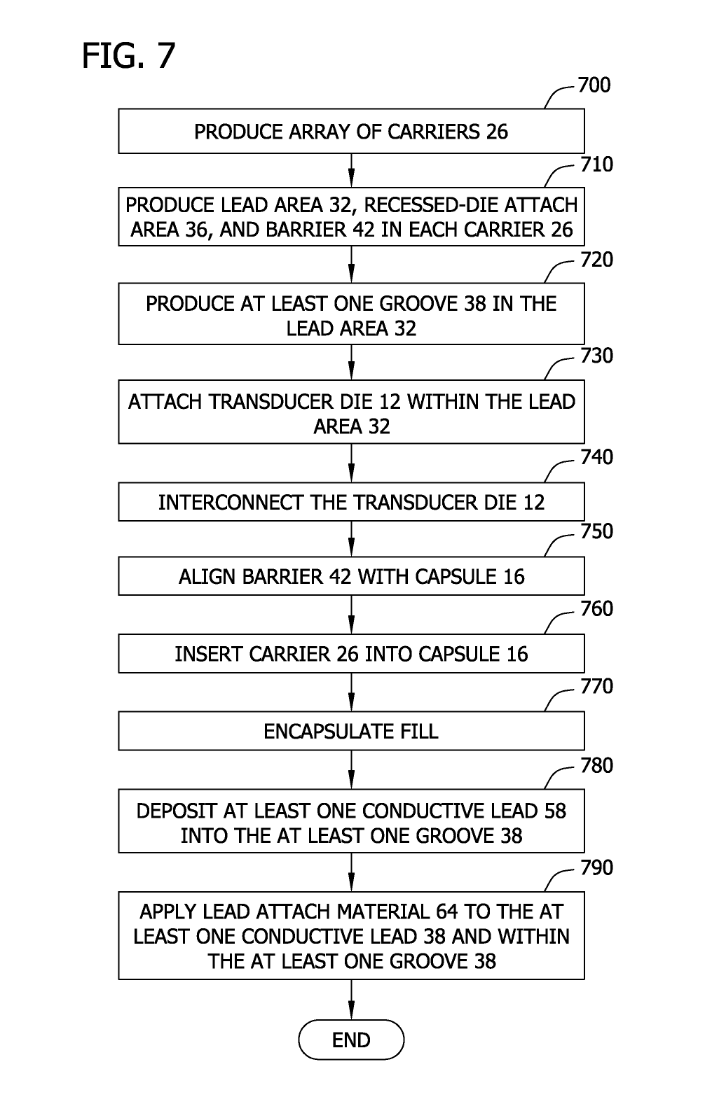

FIG. 7 is a flowchart of an exemplary method for use in manufacturing the catheter tip device 10. In the exemplary embodiment, any or all of the manufacturing process 700-790 can be fully automated, thus providing for significant quality improvement and cost reduction.

Initially, an array of carriers 26 is produced 700 using MID technology. Alternatively, any molding or machining process that can produce carrier 26 such that carrier 26 functions as described herein may be used. Each carrier 26 is formed 710 with at least one recessed die-attach area 36 that enables at least one transducer die 12 to be attached therein. Each carrier 26 is produced 710 such that a barrier 42 is positioned on at least die attach area 30 and lead pad area 34. During manufacturing, lead area 32 is produced 720 with the at least one groove 56.

At least one transducer die 12 is then attached 730 to the recessed die-attach area 36. The transducer die 12 can be attached 730 with a recessed die-attach area 36 using an adhesive agent 40, such as a silicone gel or a Room Temperature Vulcanized (RTV) silicone. This recessed die-attach area 36 enables the use of epoxy by placing the pre-formed epoxy in the recessed die-attach area 36, then placing the transducer die 12, and then reflowing the epoxy without the risk of overflow.

Each transducer die 12 is interconnected or electrically coupled 740. Once interconnected, the barrier 42 provides protection to the transducer die 12 and interconnects 66 from contacting or bumping the capsule 16 during handling, manufacturing or transportation processes.

Upon completion the interconnection of the transducer die 12, an array of completed transducer modules 104 is produced, each transducer module 14 in the array including transducer die 12 attached to carrier 26, and one or more interconnects 66 between the transducer die 12.

Next, a plurality of capsules 16 can be mounted to a fixture (not shown). The fixture can have at least one recessed area with openings configured to receive capsules 16. The capsules 16 can be provided in an array to facilitate placement into the openings of the fixture.

A side of the array of carriers 26 is removed or singulated to expose transducer module 14. A completed transducer module 14 is extracted from the array. Once extracted, barrier 42 is aligned 750 with a particular capsule 16 so that the completed transducer module 14 can be inserted into the capsule 16. During insertion, the opposing sidewalls 46 of the barrier 42 align 750 with grooves 24 of capsule 16. Sidewalls 46 are inserted adjacent 760 to grooves 24 to enable the die recessed area 36 within the cavity 18 of the capsule 16.

The inserted carrier 26 is attached to capsule 16, by a process such as, but not limited to, via plastic welding, solvent bonding, or using an adhesive agent. The capsules 16 can be filled with an encapsulant provided 770 such as, for example, by a dielectric silicone potting by an automated process to protect the transducer die 12 and interconnect 66 from the external environment.

At least one conductive lead 58 is deposited 780 within the groove 56 such that groove 56 aligns the conductive lead 58. After placing the conductive lead 58 within groove 56, lead attach material 64 is applied 790 to conductive lead 58 and within groove 56 to enable the conductive lead 58 to be attached to carrier 26. During manufacturing, groove 56 facilitates aligning the conductive lead 58 to the lead area 32. Grooves 56 also facilitate maintaining material 64 within groove 56 and on the conductive lead 58 such that the likelihood that material will electrically bridge to an adjacent conductive lead 58 is reduced.

In another method, prior to extruding the transducer module 14, conductive lead 58 is deposited within the groove 56 such that groove 56 aligns 730 the conductive lead 58. After placing the conductive lead 58 within groove 56, lead attach material 64 is applied to conductive lead 58 and within groove 56 to enable the conductive lead 58 to be attached to carrier 26. Then, the transducer module 14 is extracted by removing or singulating a side of the array of carriers 26 for insertion into the capsule 16.

The manufacturing of the catheter tip device 10 facilitates inserting transducer module 14 into capsule 16. Transducer module 14 is sized and oriented to accurately insert within capsule 16 while protecting dies, leads and associated electrical interconnections during the manufacturing processes. Accurately manufacturing transducer module 14 and inserting transducer module 14 within capsule 16 enhances the overall manufacturing of device 10 by preventing or removing difficulties and/or inaccurate handling by an assembly technician. Additionally, accurate insertion of transducer module 14 enhances operation of the catheter tip device 10 and facilitates placement of the catheter tip device 10 within the body.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A transducer module comprising:
a carrier comprising a recessed-die attach area and a lead area, said lead area comprising at least one groove formed therein; and
a barrier extending from said carrier and partially circumscribing said recessed-die attach area to facilitate protecting said recessed-die attach area during manufacturing processes.

2. The transducer module of claim 1, wherein said at least one groove extends substantially axially along said lead area.

3. The transducer module of claim 1, wherein said at least one groove facilitates aligning at least one conductive lead deposited onto said carrier.

4. The transducer module of claim 3, wherein said carrier comprises a transducer die coupled to said recessed die-attach area, the transducer die is electrically connected to said at least one conductive lead by an interconnect.

5. The transducer module of claim 4, wherein said barrier extends upward from said carrier a distance above said interconnect.

6. The transducer module of claim 1, wherein said barrier comprises a pair of opposing sidewalls and an end wall extending between said sidewalls.

7. The transducer of claim 6, wherein each sidewall comprises a first end, a second end and body extending between said first end and said second end.

8. The transducer of claim 7, wherein said first end has a height that is shorter than a height of said second end.

9. A catheter tip device comprising:
a capsule;
a transducer module attached to said capsule, said transducer module comprising:
a carrier comprising a recessed die-attach area and comprising a lead area,
a transducer die located in said recessed die-attach area, and
at least one conductive lead deposited into at least one groove of said lead area, said at least one conductive lead electrically interconnected to said transducer die; and
a barrier extending from said carrier and partially circumscribing said recessed-die attach area to facilitate protecting said recessed-die attach area during manufacturing processes.

10. The catheter tip device of claim 9, wherein said at least one groove comprises opposing walls that facilitate aligning said at least one conductive lead deposited onto said carrier.

11. The catheter tip device of claim 9, wherein said barrier comprises a pair of opposing sidewalls and an end wall.

12. The catheter tip device of claim 11, wherein said pair of opposing sidewalls partially circumscribes said electrical interconnection of said transducer die and said at least one conductive lead.

13. The catheter tip device of claim 9, wherein each sidewall comprises a first end, a second end and body extending between the first end and the second end.

14. The catheter tip device of claim 13, wherein said body is tapered between said first end and said second end.

15. A method of manufacturing a catheter tip device comprising:
forming a carrier that comprises a recessed die-attach area, a grooved lead area and a barrier circumscribing at least partially around said recessed die-attach area;
attaching at least one transducer die to the recessed-die attach area; and
interconnecting the at least one transducer die.

16. The method of claim 15, further comprising depositing at least one conductive lead comprises within said grooved lead area.

17. The method of claim 16, further comprising applying deposition material into said grooved lead area.

18. The method of claim 15, wherein forming said carrier comprises forming said barrier comprising a pair of opposing sidewalls and an end wall.

19. The method of claim 18, further comprising aligning the barrier with a capsule by aligning said pair of opposing sidewalls of said barrier into grooves of said capsule.

20. The method of claim 15, further comprising inserting the carrier within said capsule.
